(19) United States
(12) Patent Application Publication
(10) Pub. No.: US 2019/0164651 A1
(43) Pub. Date: May 30, 2019

(54) HEALTH TASK GENERATION AND POPULATION

(71) Applicant: Lightbeam Health Solutions, Inc., Irving, TX (US)

(72) Inventor: Jerry Shultz, Irving, TX (US)

(21) Appl. No.: 16/204,435

(22) Filed: Nov. 29, 2018

Related U.S. Application Data
(60) Provisional application No. 62/591,992, filed on Nov. 29, 2017.

(51) Int. Cl. G16H 80/00; G16H 50/30; G16H 10/60

(52) U.S. Cl. CPC G16H 80/00; G16H 10/60; G16H 50/30

(57) ABSTRACT

A method and system for providing one or more care provider task items to be used at a client computing machine, wherein the method includes: obtaining electronically encoded patient data from one or more data sources, wherein the electronically encoded patient data comprises information pertaining to a patient or group of patients; preparing the patient data for standardized entry into one or more of the databases using the processor of at least one of the electronic computing machines; determining a diagnostic property or condition of the patient or group of patients; obtaining care information on the diagnostic property or condition of the patient or group of patients; automatically generating a care provider task item based on the care information; and sending the care provider task item to a client computing machine via an inter-connected electronic data network.

### CROSS-REFERENCE TO RELATED APPLICATIONS

This is a U.S. non-provisional patent application claiming the benefit of priority from U.S. provisional patent application No. 62/591,992 filed on Nov. 29, 2017, the entire contents of which are incorporated herein.

### BACKGROUND

### 1. Field of the Invention

The invention relates to the field of providing auto-generated health-related task items to a client computing machine for access by a user.

### 2. Description of the Related Art

Today, health systems, healthcare providers, healthcare professionals, payers and risk-bearing entities use computer tools for viewing health records and other health-related data. Although these computer tools may facilitate entry and viewing of electronic health records and other health-related data, there is a need to automatically generate and populate an electronic health record or patient profile with health-related task information that is tailored to a specific patient or group of patients.

### SUMMARY

According to an embodiment of the invention, there is provided a method of providing one or more care provider task items to be used at a client computing machine. The method is carried out by one or more servers, wherein the one or more servers comprises at least one database stored on a computer-readable memory. At least one of the servers comprises one or more electronic computing machines, and the electronic computing machines each comprise a processor and a computer-readable memory. The method includes the steps of obtaining electronically encoded patient data from one or more data sources, wherein the electronically encoded patient data comprises information pertaining to a patient or group of patients; preparing the patient data for standardized entry into one or more of the databases using the processor of at least one of the electronic computing machines; determining a diagnostic property or condition of the patient or group of patients; obtaining care information on the diagnostic property or condition of the patient or group of patients; automatically generating a care provider task item based on the care information; and sending the care provider task item to a client computing machine via an inter-connected electronic data network, wherein the client computing machine is configured to provide a care provider task item indicator in response to receipt of the care provider task item.

According to various embodiments, this method may further include any one of the following features or any technically-feasible combination of two or more of these features:
the patient data being based at least partly on data from an electronic medical record (EMR) of the patient or group of patients;
the care provider task item being generated based at least partly on the EMR of the patient or group of patients;
the care provider task item being generated based on a past or scheduled patient action;
storing the standardized patient data into the one or more databases; and/or
the care information being obtained by querying the at least one databases or from one or more remote servers via the inter-connected electronic data network.

### BRIEF DESCRIPTION OF THE DRAWINGS

Preferred exemplary embodiments will hereinafter be described in conjunction with the appended drawings, wherein like designations denote like elements, and wherein:

FIG. 1 is a schematic drawing of an embodiment of a patient management system that is capable of carrying out the method(s) disclosed herein; and

FIG. 2 is a flowchart depicting an embodiment of a method of providing one or more care provider task items to be used at a client computing machine.

### DETAILED DESCRIPTION

The method(s) and system(s) provided herein are directed to providing one or more care provider task items to be used at a client computing machine. As mentioned above, technical problems in health-related electronic records exist in that the formats and storage locations of various health-related information are disparate and dispersed over many different platforms, and in that health care providers must themselves create tasks for each of their various patients, all of which may require different and unique treatment plans. At least according to one embodiment, the method(s) and system(s) provided herein can solve these technical problems by aggregating and standardizing health-related data from a variety of different data sources, using the standardized data to generate care information (e.g., care or treatment plans) for certain diagnoses, evaluating and/or otherwise processing data concerning each individual patient to identify one or more care provider task items for each patient, and then providing the one or more care provider task items to a client computing machine that displays a graphical user interface (GUI) accessible by a health-care provider and that automatically-populates the automatically-generated care provider task item(s) in the GUI.

While some client applications allow for viewing of health-related data from various different sources, these systems still require manual user input and maintenance. Thus, the method(s) and system(s) provided herein have been developed to aggregate and standardize health-related data from a variety of data sources, determining a diagnostic property or condition of the patient or group of patients, obtaining care information on the diagnostic property or condition of the patient or group of patients, and then automatically generating a care provider task item based on the care information. The method(s) and system(s) provided herein, in one embodiment, automatically generates a care provider task item based on a patient's particular conditions so that the health care provider can be provided with a graphical user interface (GUI) that is automatically populated with tasks (e.g., event reminders) that are to be performed by the health provider.

In some embodiments, the method(s) provided herein can obtain patient data, determine a diagnostic property or condition of a particular patient or group of patients, obtain care information based on the diagnostic property or condition, and then automatically generate a care provider task item based on the care information. As used herein, a “care provider task item” refers to an item that represents a medical-related or health-related task and/or event, such as prescription orders and renewals, provider visitation dates, treatment dates, etc.

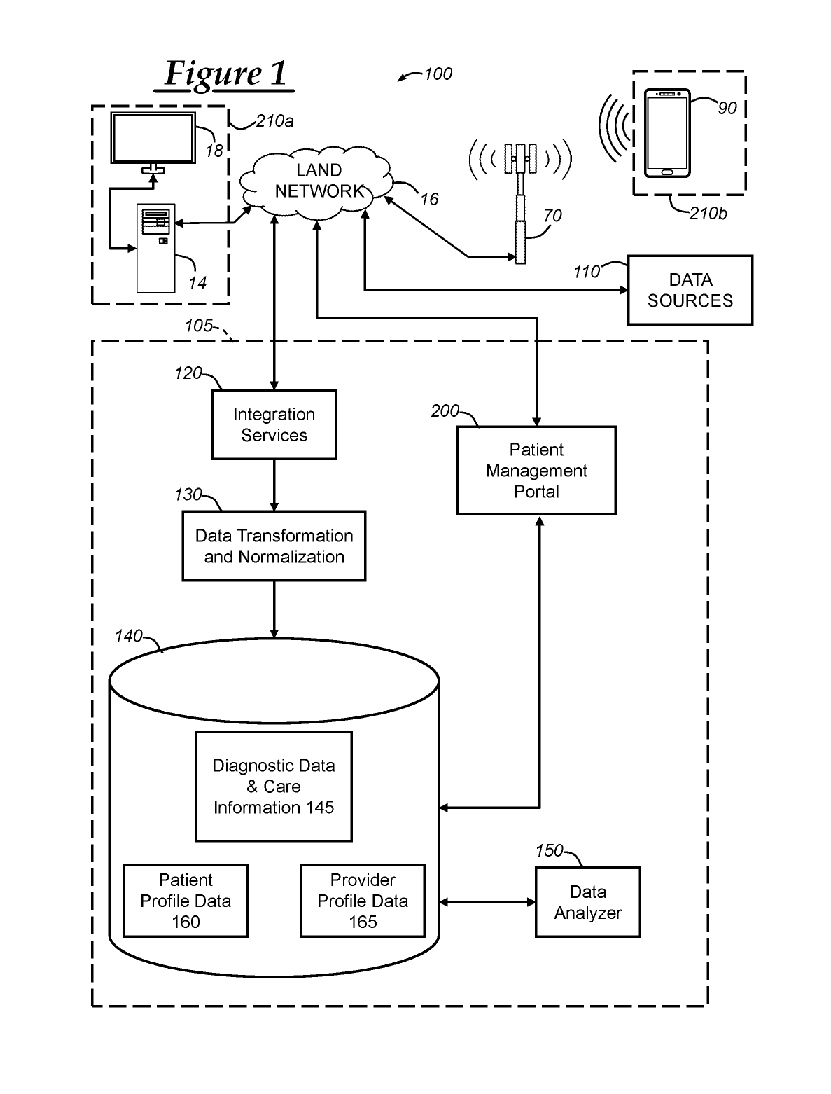

FIG. 1 represents one embodiment of a health task generation system 100 that is capable of carrying out the method disclosed herein. The system 100 includes servers 105, data sources 110, land network 16, cellular carrier system 70, and clients 210a,b. Health task generation system 100 can work completely on a stand-alone basis or with aggregated data from third party data sources 110 (only one shown). The one or more servers 105 (only one shown) may carry out the method provided herein and each server may, at least in some embodiments, include a plurality of electronic computing machines and at least one database 140 stored on a computer-readable memory. As used herein, an electronic computing machine may be a computer, processing system, or other computing device capable of carrying out computer instructions.

Health task generation system 100 is configured to gather broad and discrete data on a real time basis or in batch from multiple data sources 110. The data can then be normalized and stored into database 140 at servers 105. The data can be analyzed, processed, packaged, and sent by patient management portal 200 to a client computing machine 14 or 90. The client can provide a health task generation tool that can be supported by patient management portal 200. The system 100 is capable of operating on a “standalone basis” and without data gathered from data sources 110, integrated by integration services 120 and transformed by data manipulator 130. Users 210a,b can create and maintain patient 160 and provider 165 profiles using the visual selection referral portal 200. The system 100 and its individual components will be explained in greater detail below.

Data sources 110 may be located at a location remote from the location of the one or more servers. In such an embodiment, the data sources 110 may be accessible by the integration services 120 (or other component of the servers) via land network 16 that includes an inter-connected data network, such as the Internet. Alternatively, the data sources may be stored at servers 105 on a non-volatile computer-readable medium, such as ROM (including any or all of its variants—e.g., EEPROM), a compact disc (CD), or a flash drive. The data sources may then be locally connected to the one or more servers or database.

Data sources 110 can include various information, such as electronic medical records databases (EMRs), physician and provider databases, claims, ambulatory systems, electronic health record databases (EHRs), inpatient systems, health information exchanges (HIEs), payers, care management systems, third party analytics systems, prescription data from pharmacies, Rx aggregators and pharmacy benefit managers (PBMs), laboratories, patient biometric monitoring devices, genomic databases, provider rating services, patient satisfaction, and survey data. Any data that upon observation could be related to determining provider performance and/or patient default preferences may be assembled into a consumable format by the system 100. The integration services 120 and data transformation and normalization 130 gather and prepare any data that upon observation could be used in a health task generation process.

Data is then stored in one or more databases 140 (only one shown) at one or more servers 105 (only one shown). The data may be stored in one or more tables, partitions, or databases based on the type of data or the source of the data. For example, patient data, which is data relating to patient information regarding one or more patients, may be stored in a table corresponding to the patient profile data 160. Patient management portal 200 may provide information from database(s) 140 to users 210a,b that are operating an electronic client computing machine via an inter-connected electronic data network, which may include the cloud, Internet, TCP/IP connections, VPN, and other types of connections including mobile devices using Android™, iOS™, Windows™ Mobile and Blackberry™.

Patient 160 and provider 165 profile data can also be assembled from data gathered from multiple sources 110 including but not limited to claims, ambulatory systems, EHRs, inpatient systems, electronic medical records, HIE's, payers, care management systems, LIGHTBEAM's Analytics Platform, third party analytics systems, prescription data from pharmacies, Rx aggregators and PBMs, labs, patient biometric monitoring devices, genomic databases, search engines, provider rating services, patient satisfaction and survey data.

In some embodiments, integration services layer 120 provides the transport infrastructure and supports current and future standards based interfaces such as HL7, CCD, CCR, XML, SOA, custom APIs, claims, and where applicable the IRE profiles used to integrate such as xdr, xds.b, etc. Integration services 120 may also support custom interfaces, capable of reaching directly into source systems to extract data. In one embodiment, integration servers 120 may comprise a computer application running on an electronic computing machine that is configured to download data from one or more data sources 110 using land network 16. In such an embodiment, the servers 105 may receive electronically encoded data from one or more data sources. The data may comprise information pertaining to a patient and/or a medical provider. Then, upon download of the data, integration services 120 may use an application to process the data and then pass the data to data transformation and normalization 130.

In some embodiments, the health task generation system uses data transformation and normalization 130 to map, transform and normalize data from data sources 110 and store the mapped, transformed, and normalized data in database 140. Therefore, in such an embodiment, users of the patient management tool are able to compare performance, outcomes, quality, utilization, cost and satisfaction data from third party sources and quality measures, as well as review patient data and care provider task items. The data transformation and normalization may be carried out by one or more electronic computing machines. In such an embodiment, the data transformation and normalization 130 may be a set of computer instructions that are carried out on the electronic computing machine(s) as part of a set of software instructions (i.e., an application). In one embodiment, the integration services 120 and data transformation and normalization are separate applications carried out on the same computing machine; however, one skilled in the art will appreciate that there are many different software and hardware configurations enabling such processes to be carried out.

Data transformation and normalization 130 may be used to facilitate the mapping of data from data sources using various terminology, codes, and abbreviations into a normalized data set within the database 140. The normalization will facilitate the execution of filtering on the provider dataset and may allow the data to be combined with other data in a manner as to allow care provider task item generation. In one embodiment, data transformation and normalization 130 prepares the received data for standardized entry into one or more of the databases using the processor of at least one of the electronic computing machines located at server(s) 105. As used herein, “standardize” includes mapping, transforming, and/or normalizing. The database 140 stores diagnostic data and care information, as well as patient data that can be gathered from sources 110. By statistically referencing, cross referencing, and/or analyzing the diagnostic information for patients and populations against combinations of the patient information gathered and organized in the database 140, the system may be able to identify the specific care provider tasks for each patient or group of patients. Tables in database 140 may store these care tasks.

The data is linked to a provider by the data transformation and normalization 130, and stored in database 140. In some embodiments, data analyzer 150 is used to calculate performance metrics, including scores or grades, that will be stored in provider profile 165 and displayed by the patient management tool through operation of patient management portal 200. A performance metric of a provider may include any data concerning past performance by the provider, including any provider reviews, patient satisfaction ratings, costs, provider quality scores, and quality of services or goods. Data analyzer 150 evaluates data stored in the diagnostic data and care information table 145, patient profile data 160, and/or provider profile data 165. In one embodiment, data analyzer 150 is carried out at server(s) 105 and comprises a set of computer instructions carried out on one or more processors of one or more electronic computing machines. Additionally, data analyzer is electronically coupled to database 140 such that it may query database 140 and/or perform other database operations thereon, such as performing insertion, modification, and/or deletion operations on data tuples, tables, or other data structures. Data analyzer 150 may use computerized database interfacing software, such as SQL, MySQL™, Microsoft™ SQL Server, and/or other suitable database management systems (DBMS).

In some embodiments, criteria used to determine a diagnostic property or condition of a patient or a group of patients may be based on historical data regarding the patient's or group of patients' particular medical condition, as well as the patient's or group of patients' past medical history, which can include provider visits. The data analyzer 150 may determine the diagnostic property or condition and determine care information based on such diagnostic property or condition and/or based on a patient's past medical history, family medical history, other patient data, and/or the patient's provider visitation history. Additionally, or alternatively, the data analyzer 150 may determine a care provider task item based on the care information, diagnostic information, patient data, and/or or provider data.

Patient management portal 200 may be used to provide computer instructions or data to a client machine that can visually represent patient data, diagnostic data, provider data, and/or care provider task items graphically on the client machine. Indicators may be used to graphically represent such information. The patient management portal 200 can be a web-server that responds to HTTP/HTTPS request or other data request from the client machine. In one embodiment, instructions constituting a web application may be served by the portal 200 to client devices 210a,b and, upon receipt, the client devices may visually display the application. In another embodiment, an application can be installed on client device 210a,b and, in such cases, the portal 200 may serve information for use in the application.

When the user 210 wants to use the patient management tool, the user can execute the software application or initiate a web application request (e.g., via a web browser that is installed on the client device 210). The client device will then trigger a data request that can be received at the patient management portal 200 via the land network 16 and/or cellular carrier system 70. In one embodiment, the user would operate computer 14 or mobile device 90 such that a client request for access to the patient management tool is generated and sent to server(s) 105. One or more electronic computing machines at server 105 may then receive the client request via an inter-connected electronic data network, such as land network 16 and/or cellular network 70. The user may invoke generation and sending of the client request via navigation to a specified website or IP address, which resolves to one or more devices located at server 105. The navigation may be carried out via an Internet or network browser on computer 14 or mobile device 90; alternatively, a computer application or mobile application may be operated by the user wherein the application generates and sends such a client request.

After the server 105 receives the client request, the client request may be resolved to a user. For example, the client request may include a user identifier, username, or other identifying information that associates a user with the patient management tool. After the client request is resolved to a user, the client request may be processed by querying at least one database for patient/provider information pertaining to the user, to a patient of the user, to a provider of the user, or to a potential provider of the user, using a visual selection referral application installed on one or more of the electronic computing machines. In one embodiment where the user is a medical provider, the database 140 may be queried to retrieve data from patient profile data 160, provider profile data 165, and/or diagnostic data & care information 145. Any or all of this data may be particular to the user and/or may filtered down based on other parameters relating to the client request that was received at server 105. For example, patient profile data 160 may be queried for information pertaining to one or more patients or potential patients of the user. The foregoing processing of the client request may be carried out by a patient management portal 200 located at server 105. Patient management portal 200 may be a computer application installed on one or more electronic computing machines each comprising a processor and a computer-readable memory.

After the client request is processed, a response is generated and sent to the electronic client computing machine over the inter-connected electronic data network. The response may be generated and sent by visual selection referral portal 200 and may include patient/provider information that is usable for the medical visual referral tool at the client computing machine. The response may include data that was retrieved from database 140 or may include data or information that was generated based on data retrieved from the database 140.

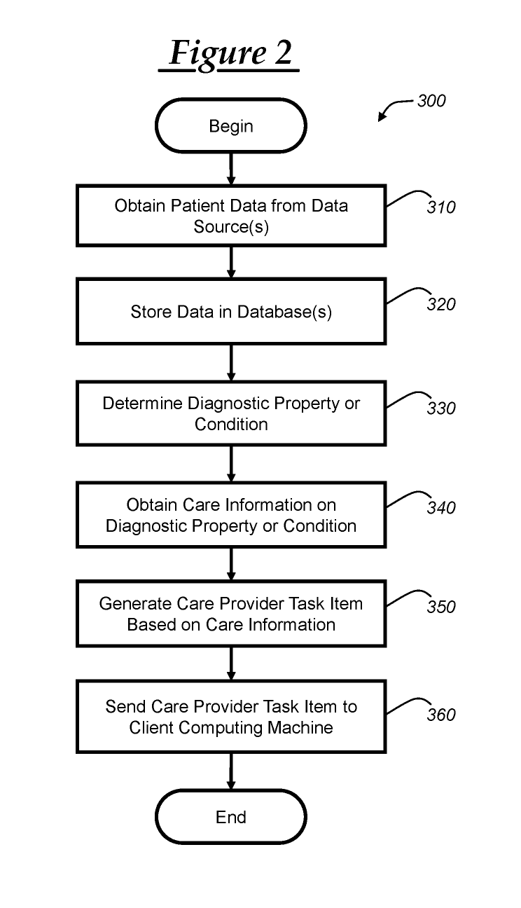

FIG. 2 depicts an embodiment of a method 300 of providing one or more care provider task items to be used at a client computing machine. The method 300 includes: a data fetching and standardization process (steps 310 to 320); and a task item generation process (steps 330 to 360). In many embodiments, the method 300 may be carried out by one or more servers, wherein the one or more servers comprises at least one database stored on a computer-readable memory. Additionally, at least in some embodiments, each of the servers comprises a plurality of electronic computing machines, wherein the electronic computing machines each comprise a processor and a computer-readable memory.

The method 300 begins with step 310 wherein one or more servers receive electronically encoded data from one or more data sources. In some embodiments, the electronically encoded data comprises information pertaining to one or more patients, one or more groups of patients, and/or one or more medical providers. In one embodiment, the data is gathered by integration services 120. In such an embodiment, integration services 120 may be a computer application running on one or more servers and configured to download data from one or more data sources 110 using an inter-connected data network, such as land network 16 and/or cellular network 70. In another embodiment, data may be located and/or retrieved from the data sources in response to the server receiving a client request. Furthermore, the data located and/or retrieved may be based on information included in the client request.

In some embodiments, once the data is received, the data is prepared for standardized entry into one or more of the databases using the processor of at least one of the electronic computing machines. As described above, data transformation and normalization 130 may be a process or application carried out one an electronic computing machine. In some embodiments, the data transformation and normalization 130 is carried out on the same processor and/or as part of the same process or application as integration services 120. As is also described above, data transformation and normalization may map, transform, normalize, standardize, and/or process the data received at step 310 such that it is in a proper format usable by the medical visual referral tool. In addition, the data may be formatted such that it will be suitable for insertion into one or more databases 140. In an alternative embodiment, the database may comprise one or more data files, such as XML files, JSON files, and/or comma-separated value files, that are stored on a computer-readable medium. After the data is downloaded, the method 300 proceeds to step 320.

In step 320, the standardized data is stored in at least one database. In one embodiment, a database management application may carry out one or more insertion operations on database 140. Thereafter, the database management application may carry out a commit operation, such that the insertion is committed to the database(s) 140. After the data is committed to the database, the patient management portal may be used to query the database, which now includes the newly committed data, for use in hosting the medical visual referral tool. Alternatively, or additionally, the new data can be used in the automatic generation of a care provider task item, as will be discussed in more detail below. The method 300 continues to step 330.

In step 330, a diagnostic property or condition of the patient or group of patients is determined. The diagnostic property or condition can be determined based on the patient data and/or provider data. For example, a patient's present medical condition (e.g., broken leg, diabetes) may be used to develop a patient care schedule and/or plan, which may be kept in patient profile data 160. One or more patients may have similar medical conditions and, in some embodiments, the patient management portal 200 may determine diagnostic information for a group of patients. The diagnostic information can be a typical schedule of events and/or tasks that should be performed for a patient whom suffers from a given medical condition, which can include check-up dates (e.g., periods in which the patient should go to the care provider's office for examination) and/or prescription medicines. Once the diagnostic property or condition is determined, then the method 300 continues to step 340.

In step 340, care information on the diagnostic property or condition of the patient or group of patients is obtained. The care information can be information that relates to a customized schedule for a given patient based on the diagnostic information (see step 330), the patient's medical history (including the patient's family medical history), the patient's past visitations to care providers, and/or the patient's past medication prescription orders. The care information can use this information to provide customizable and personalized care information for a given patient. The data analyzer 150, patient management portal 200, and/or any other application may obtain this information through data processing and/or analysis. The method 300 then continues to step 350.

In step 350, a care provider task item based on the care information is automatically generated. As mentioned above, a care provider task item may be any item that represents a medical- and health-related tasks and/or events, such as prescription orders and renewals, due dates for a follow-up appointment, etc. The task item may be electronically represented data, such as an XML, JSON, or HTML element, that is then sent to the client device (in step 360). The item may be generated by portal 200 and may be sent automatically to the health task generation tool. The method 300 continues to step 360.

In step 360, the care provider task item is sent to a client computing machine via an inter-connected electronic data network. In many embodiments, the client computing machine is configured to provide a care provider task item indicator in response to receipt of the care provider task item. The task item may be sent to client devices via land network 16 and/or cellular carrier system 70. In some embodiments, the task item may be sent using HTTP/HTTPS, FTP, and/or various other packet-switched data transfer protocols.

Once the health task generation tool receives the task item, the tool may present the task item using a graphical user interface (GUI) in the form of a graphical icon or object that conveys to the user that a care provider task should soon be carried out or is to be soon carried out. The GUI may be presented on display 18 that is connected to client computing machine 14, or on a visual display of client computing machine 90. The graphical object may include numerous information that can be displayed in graphical form, such as a due date, a patient's name, the subject of the task item, and the description of the task item. Various other information can be presented in other embodiments. Also, when a particular task item is selected or highlighted by a user, other windows of the GUI can show more detailed information. The client device can use other human machine interfacing means to convey this task item, such as through use of an audio system. The method 300 then ends.

The system and method have been described in an illustrative manner. It is to be understood that the terminology, which has been used, is intended to be in the nature of words of description rather than of limitation.

Many modifications and variations of the system and method are possible in light of the above teachings. Therefore, within the scope of the appended claims, the system and method may be practiced other than as specifically described.

We claim:

1. A method of providing one or more care provider task items to be used at a client computing machine, the method being carried out by one or more servers, wherein the one or more servers comprises at least one database stored on a computer-readable memory, wherein at least one of the servers comprises one or more electronic computing machines, wherein the electronic computing machines each comprise a processor and a computer-readable memory, and wherein the method comprises:
(a) obtaining electronically encoded patient data from one or more data sources, wherein the electronically encoded patient data comprises information pertaining to a patient or group of patients;
(b) preparing the patient data for standardized entry into one or more of the databases using the processor of at least one of the electronic computing machines;
(c) determining a diagnostic property or condition of the patient or group of patients;
(d) obtaining care information on the diagnostic property or condition of the patient or group of patients;
(e) automatically generating a care provider task item based on the care information; and
(f) sending the care provider task item to a client computing machine via an inter-connected electronic data network, wherein the client computing machine is configured to provide a care provider task item indicator in response to receipt of the care provider task item.

2. The method of claim 1, wherein the patient data is based at least partly on data from an electronic medical record (EMR) of the patient or group of patients.

3. The method of claim 2, wherein the care provider task item is generated based at least partly on the EMR of the patient or group of patients.

4. The method of claim 3, wherein the care provider task item is generated based on a past or scheduled patient action.

5. The method of claim 1, further comprising the step of storing the standardized patient data into the one or more databases.

6. The method of claim 1, wherein the care information is obtained by querying the at least one databases or from one or more remote servers via the inter-connected electronic data network.
